(19) United States
(12) Patent Application Publication
(10) Pub. No.: US 2017/0065360 A1
(43) Pub. Date: Mar. 9, 2017

(54) Illumination System

(71) Applicant: Christopher Nichols, Tacoma, WA (US)

(72) Inventor: Christopher Nichols, Tacoma, WA (US)

(21) Appl. No.: 14/845,690

(22) Filed: Sep. 4, 2015

(51) Int. Cl. A61B 90/00; F21V 23/02; F21V 21/088

(52) U.S. Cl. CPC A61B 90/36; F21V 21/088; F21V 23/023; F21W 2131/205

(57) ABSTRACT

An illumination system for providing disposable, sterile illumination includes a surgical instrument that may be manipulated to perform a surgical procedure. A lighting unit is removably coupled to the surgical instrument such that the lighting unit illuminates a work area with respect to the surgical instrument. The lighting unit is sterile such that the lighting unit may be utilized in a sterile environment.

### BACKGROUND OF THE DISCLOSURE

### Field of the Disclosure

The disclosure relates to illumination devices and more particularly pertains to a new illumination device for providing disposable, sterile illumination.

SUMMARY OF THE DISCLOSURE

An embodiment of the disclosure meets the needs presented above by generally comprising a surgical instrument that may be manipulated to perform a surgical procedure. A lighting unit is removably coupled to the surgical instrument such that the lighting unit illuminates a work area with respect to the surgical instrument. The lighting unit is sterile such that the lighting unit may be utilized in a sterile environment.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of an illumination system according to an embodiment of the disclosure.

FIG. 2 is a front view of an embodiment of the disclosure.

FIG. 3 is a bottom view of an embodiment of the disclosure.

FIG. 4 is a cross sectional view taken along line 4-4 of FIG. 2 of an embodiment of the disclosure.

FIG. 5 is a perspective in-use view of an embodiment of the disclosure.

FIG. 6 is a perspective view of an alternative embodiment of the disclosure.

### DESCRIPTION OF THE PREFERRED EMBODIMENT

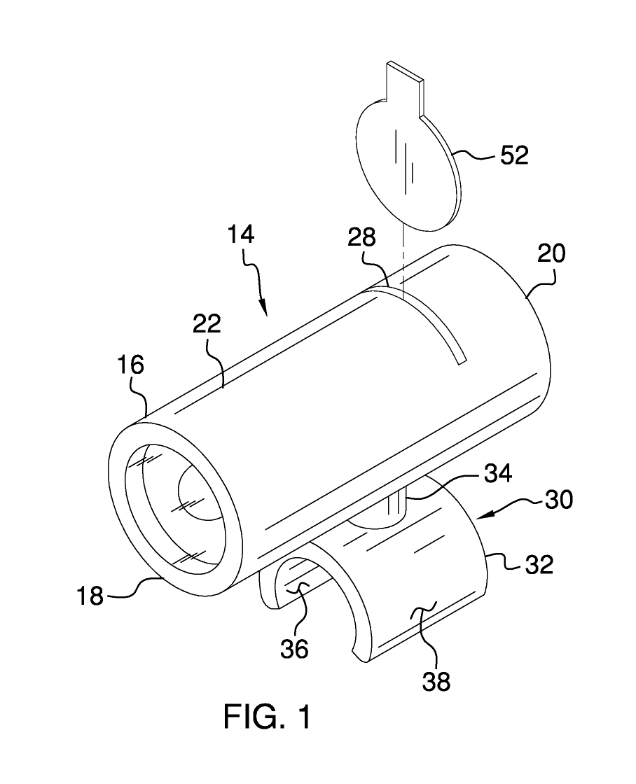

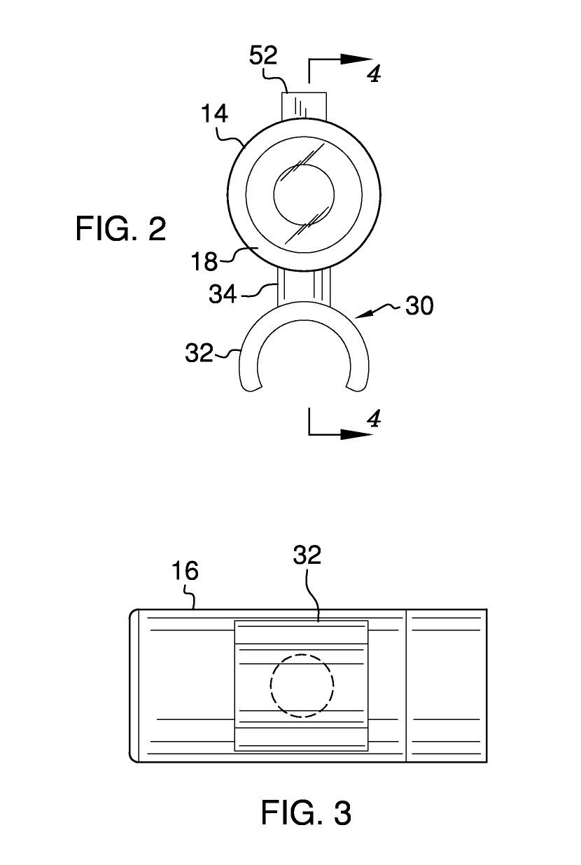

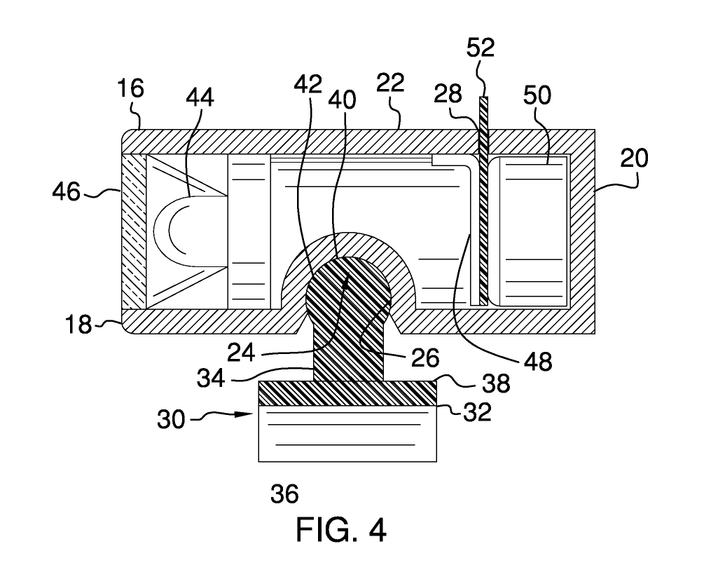

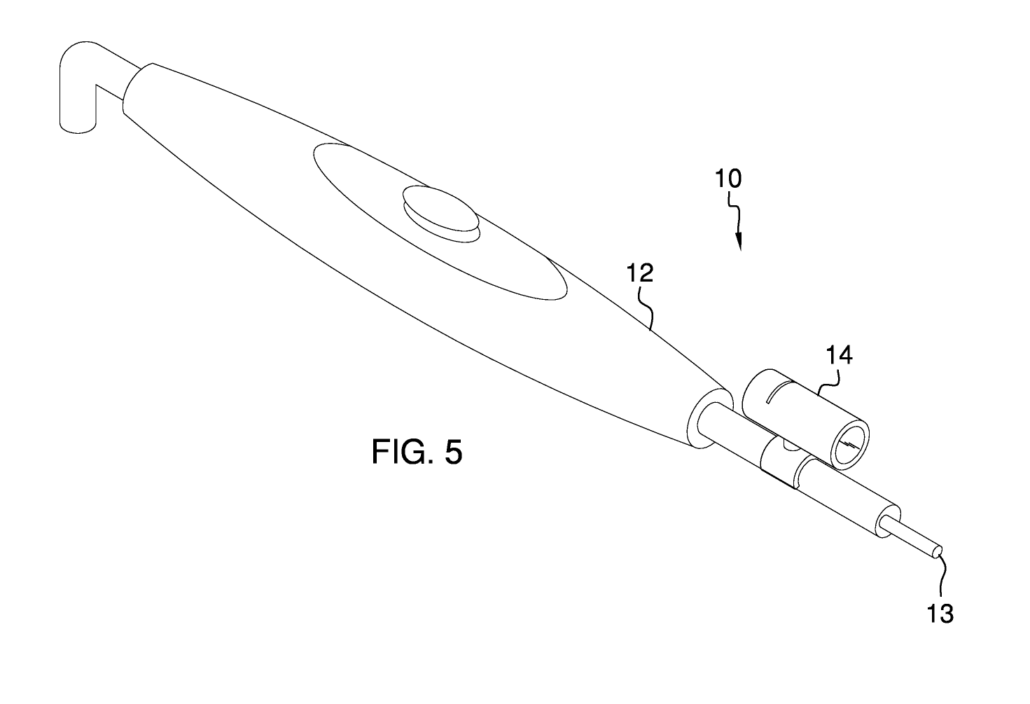

With reference now to the drawings, and in particular to FIGS. 1 through 6 thereof, a new illumination device embodying the principles and concepts of an embodiment of the disclosure and generally designated by the reference numeral 10 will be described.

As best illustrated in FIGS. 1 through 6, the illumination system 10 generally comprises a surgical instrument 12 that has a primary end 13. The surgical instrument 12 may be manipulated during a surgical procedure. The surgical instrument 12 may be a hand held surgical instrument of any conventional design. A lighting unit 14 is removably coupled to the surgical instrument 12 such that the lighting unit 14 illuminates a work area with respect to the surgical instrument 12. The lighting unit 14 is sterile such that the lighting unit 14 may be used in a sterile environment. The sterile environment may be an operating room and the lighting unit 14 is stored prior to use in any sterile container utilized in the convention of surgical procedures.

The lighting unit 14 comprises a housing 16 that has a first end 18, a second end 20 and a peripheral wall 22 extending between the first end 18 and the second end 20. The peripheral wall 22 is curved such that the housing 16 has a cylindrical shape. The first end 18 is open and the peripheral wall 22 has a well 24 extending inwardly therein. The well 24 has a bounding surface 26 and the bounding surface 26 is curved such that the well 24 has a hemispherical shape. The well 24 is centrally positioned between the first end 18 and the second end 20. The peripheral wall 22 has a slot 28 extending therethrough and the slot 28 is positioned proximate the second end 20.

A mount 30 is provided and the mount 30 comprises a saddle 32 and a rod 34. The saddle 32 has a bottom surface 36 and a top surface 38. The bottom surface 36 is curvilinear with respect to the top surface 38 such that the saddle 32 has a parabolic U-shape. The rod 34 extends upwardly from the top surface 38. The rod 34 has a distal end 40 with respect to the saddle 32 and the distal end 40 is bulbous such that the distal end 40 forms a ball 42. The ball 42 is positioned within the well 24 such that the housing 16 is rotatably coupled to the mount 30.

The saddle 32 frictionally engages the surgical instrument 12. Thus, the mount 30 is removably retained on the surgical instrument 12. The first end 18 of the housing 16 is directed toward the primary end 13 of the surgical instrument 12. A light emitter 44 is positioned within the housing 16. The light emitter 44 is positioned adjacent to the first end 18 and the light emitter 44 selectively emits light outwardly from the first end 18. The light emitter 44 may comprise an LED or the like. A lens 46 is coupled to the first end 18 such that the lens 46 completely covers the first end 18.

A contact 48 is positioned within the housing 16. The contact 48 is electrically coupled to the light emitter 44 and the contact 48 is aligned with the slot 28. A power supply 50 is positioned within the housing 16. The power supply 50 is positioned adjacent to the second end 20 such that power supply 50 is aligned with the slot 28.

A pad 52 is removably positioned within the slot 28. The pad 52 extends upwardly from the slot 28 thereby facilitating the pad 52 to be gripped. The pad 52 extends between the contact 48 and the power supply 50. The pad 52 is comprised of an electrically insulating material. Thus, the pad 52 interrupts electrical communication between the power supply 50 and the contact 48. The pad 52 is removed from the slot 28 such that the power supply 50 is placed in electrical communication with the contact 48.

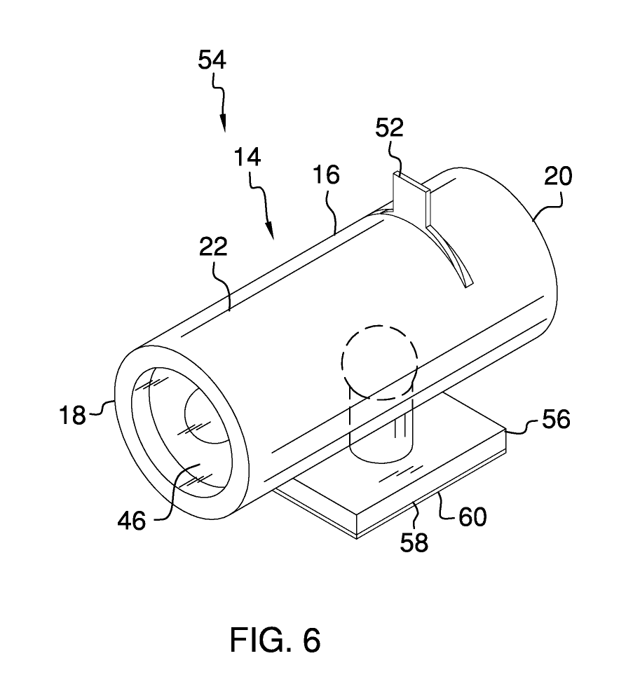

In an alternative embodiment 54 as shown in FIG. 6, the mount 30 may comprise a plate 56 and the rod 34. The plate 56 has a bottom side 58 and an adhesive layer 60 is coupled to the bottom side 58. The adhesive layer 60 adhesively engages the surgical instrument 12 such that the lighting unit 14 is removably retained on the surgical instrument 12. The alternative embodiment 54 is utilized to facilitate a surgical instrument 12 that does not have a substantially cylindrical shape such as a retractor or the like.

In use, the lighting unit 14 is removed from the sterile container and the mount 30 is coupled to the surgical instrument 12. The pad 52 is gripped and the pad 52 is removed from the slot 28. Thus, the light emitter 44 begins to emit light. The housing 16 is manipulated on the rod 34 to direct the light in a desired direction. The lighting unit 14 is removed from the surgical instrument 12 when the surgical process is completed and the lighting unit 14 is discarded.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An illumination system comprising:
a surgical instrument being configured to be manipulated to perform a surgical procedure; and
a lighting unit being removably coupled to said surgical instrument wherein said lighting unit is configured to illuminate a work area with respect to said surgical instrument, said lighting unit being sterile wherein said lighting unit is configured to be utilized in a sterile environment.

2. The system according to claim 1, wherein said lighting unit comprises a housing having a first end, a second end and a peripheral wall extending between said first end and said second end, said peripheral wall being curved such that said housing has a cylindrical shape, said first end being open, said peripheral wall having a well extending inwardly therein.

3. The system according to claim 2, wherein said well has a bounding surface, said bounding surface being curved such that said well has a hemispherical shape, said well being centrally positioned between said first end and said second end, said peripheral wall having a slot extending therethrough, said slot being positioned proximate said second end.

4. The system according to claim 1, further comprising a mount, said mount comprising a saddle and a rod, said saddle having a bottom surface and a top surface, said bottom surface being curvilinear with respect to said top surface such that said saddle has a U-shape, said rod extending upwardly from said top surface.

5. The system according to claim 4, further comprising:
a housing, said housing having a well and a first end; and
said rod having a distal end with respect to said saddle, said distal end being bulbous such that said distal end forms a ball, said ball being positioned within said well such that said housing is rotatably coupled to said mount, said saddle frictionally engaging said surgical instrument such that said mount is removably retained on said surgical instrument having said first end of said housing being directed toward said primary end of said surgical instrument.

6. The system according to claim 5, further comprising:
a light emitter being positioned within said housing, said light emitter being positioned adjacent to said first end wherein said light emitter is configured to selectively emit light outwardly from said first end; and
a lens being coupled to said first end such that said lens completely covers said first end.

7. The system according to claim 6, further comprising:
said housing having a slot; and
a contact being positioned within said housing, said contact being electrically coupled to said light emitter, said contact being aligned with said slot.

8. The system according to claim 7, further comprising a power supply being positioned within said housing, said power supply being positioned adjacent to said second end such that power supply is aligned with said slot.

9. The system according to claim 1, further comprising a pad being removably positioned within said slot, said pad extending between said contact and said power supply such that said pad interrupts electrical communication between said power supply and said contact, said pad being removed from said slot such that said power supply is placed in electrical communication with said contact.

10. An illumination system comprising:
a surgical instrument having a primary end, said surgical instrument being configured to be manipulated during a surgical procedure; and
a lighting unit being removably coupled to said surgical instrument wherein said lighting unit is configured to illuminate a work area with respect to said surgical instrument, said lighting unit being sterile wherein said lighting unit is configured to be utilized in a sterile environment, said lighting unit comprising:
a housing having a first end, a second end and a peripheral wall extending between said first end and said second end, said peripheral wall being curved such that said housing has a cylindrical shape, said first end being open, said peripheral wall having a well extending inwardly therein, said well having a bounding surface, said bounding surface being curved such that said well has a hemispherical shape, said well being centrally positioned between said first end and said second end, said peripheral wall having a slot extending therethrough, said slot being positioned proximate said second end,
a mount comprising a saddle and a rod, said saddle having a bottom surface and a top surface, said bottom surface being curvilinear with respect to said top surface such that said saddle has a U-shape, said rod extending upwardly from said top surface, said rod having a distal end with respect to said saddle, said distal end being bulbous such that said distal end forms a ball, said ball being positioned within said well such that said housing is rotatably coupled to said mount, said saddle frictionally engaging said surgical instrument such that said mount is removably retained on said surgical instrument having said first end of said housing being directed toward said primary end of said surgical instrument,
a light emitter being positioned within said housing, said light emitter being positioned adjacent to said first end wherein said light emitter is configured to selectively emit light outwardly from said first end,
a lens being coupled to said first end such that said lens completely covers said first end,
a contact being positioned within said housing, said contact being electrically coupled to said light emitter, said contact being aligned with said slot,
a power supply being positioned within said housing, said power supply being positioned adjacent to said second end such that power supply is aligned with said slot, and
a pad being removably positioned within said slot, said pad extending between said contact and said power supply such that said pad interrupts electrical communication between said power supply and said contact, said pad being removed from said slot such that said power supply is placed in electrical communication with said contact.
